(19) United States
(12) Patent Application Publication
(10) Pub. No.: US 2026/0008792 A1
(43) Pub. Date: Jan. 8, 2026

(54) PROCESSES AND COMPOUNDS

(71) Applicant: RADIUS PHARMACEUTICALS, INC., Boston, MA (US)

(72) Inventor: Michael MARKEY, Northborough, MA (US)

(21) Appl. No.: 19/324,847

(22) Filed: Sep. 10, 2025

Related U.S. Application Data
(62) Division of application No. 17/430,248, filed on Aug. 11, 2021, now Pat. No. 12,441,745.
(60) Provisional application No. 62/804,391, filed on Feb. 12, 2019.

(51) Int. Cl. C07F 5/02; C07C 213/08; C07C 217/04; C07C 231/12; C07C 233/25; C07C 235/78; C07C 237/20

(52) U.S. Cl. CPC C07F 5/025; C07C 213/08; C07C 217/04; C07C 231/12; C07C 233/25; C07C 235/78; C07C 237/20

(57) ABSTRACT

Useful processes of preparation and intermediates useful for the preparation of Compound 1, a selective estrogen receptor alpha (ERα) modulator/degrader (SERM/SERD), having utility for the treatment of ER+ cancers including breast cancer are described.

### CROSS-REFERENCE TO RELATED APPLICATIONS

This application claims priority under 35 U.S.C. § 119(e) to U.S. Provisional Application 62/804,391 filed on Feb. 12, 2019. The disclosure of this prior application is considered part of the disclosure of this application and is hereby incorporated by reference it its entirety.

### FIELD OF THE INVENTION

Useful processes of preparation and intermediates useful for the preparation of Compound 1, a selective estrogen receptor alpha (ERα) modulator/degrader (SERM/SERD), having utility for the treatment of ER+ cancers including breast cancer are described.

### BACKGROUND

Breast cancer is the second leading cause of cancer-related death in women, with an estimated 246,660 newly diagnosed cases and 40,450 deaths in the United States alone in 2016. Breast cancer is a heterogeneous disease divided into three subtypes based on expression of three receptors: estrogen receptor (ER), progesterone receptor (PR), and human epidermal growth factor receptor-2 (Her2). Overexpression of ERs is found in many breast cancer patients. ER-positive (ER+) breast cancers comprise two-thirds of all breast cancers. Other than breast cancer, estrogen and ERs are associated with, for example, ovarian cancer, colon cancer, prostate cancer and endometrial cancer.

Compound 1 has demonstrated promising mixed activity as a selective estrogen receptor alpha (ERα) modulator/degrader (SERM/SERD) against ER+ breast cancer, acting as a SERM at low doses and a SERD at high doses. In response to the growing demand for Compound 1, more efficient syntheses are required to provide increased quantities of Compound 1 to be used in additional clinical studies and potential future commercial use.

### SUMMARY OF THE INVENTION

In certain embodiments, compounds (a)-(g) are useful in the preparation of a selective estrogen receptor modulator/degrader as disclosed herein.

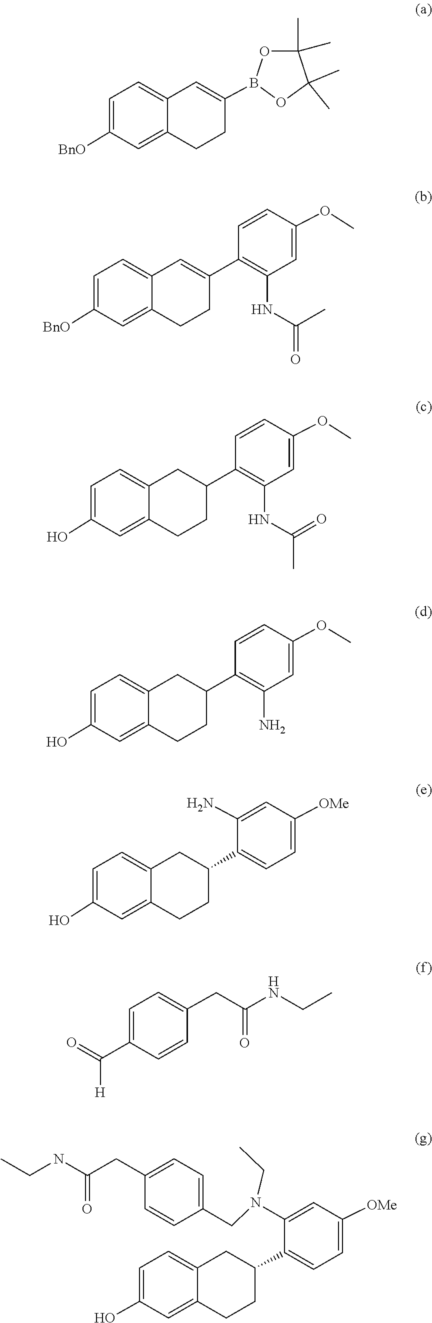

In certain aspects, the compounds of formula (e) and (g) are present with an enantiomeric excess >50%, 60%, 70%, 80%, 90%, 95%, 98%, 99%. In some embodiments, the compound having formula (e) or (g) is present in an enantiomeric excess of >50%.

In several embodiments, processes useful in the preparation of intermediates and in the preparation of Compound 1 are provided.

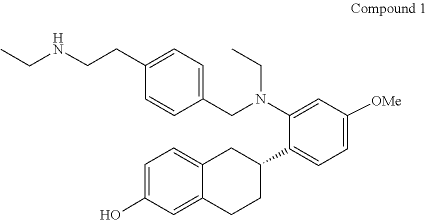 [SMILES: CCNCCc1ccc(CN(CC)c2cc(OC)ccc2[C@@H]2CCc3cc(O)ccc3C2)cc1]

In one embodiment, a process for the preparation of a compound of formula (II) is provided:

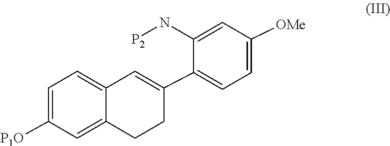

comprising the reaction of a compound of formula (I):

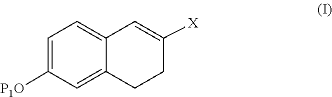

with a compound of formula (II):

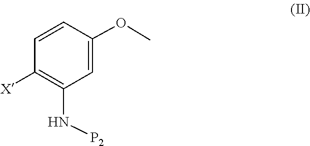

in the presence of a base and a transition metal catalyst;
wherein P₁ is H or a phenol protecting group, P₂ is H, Et or an amino-protecting group, X is a halogen, transition metal or boron-containing compound and X′ is a halogen, transition metal-containing function or boron-containing function wherein said X and X′ are suitable for cross-coupling of compound (I) with compound (II).

In some embodiments of the process for the preparation of the compound of formula (III), P₁ is H, (C═O)—C₁-C₈ alkyl, (C═O)-aryl, (C—O)-heteroaryl, Si(C₁-C₅ alkyl)₃, Si(aryl)₂(C₁-C₅ alkyl) or CH₂Aryl; P₂ is H, Et, or an amino protecting group selected from (C═O)—C₁-C₈ alkyl, (C═O)-aryl, (C═O)-heteroaryl, (C═O)—O—C₁-C₈ alkyl, (C═O)—O-aryl, (C═O)—O-heteroaryl, (C═O)—O—C₁-C₈ alkylaryl and (C═O—(CH₂)ₙ—C═O)—; X is B(OR)₂, B(—O—(C(Rₐ)₂)ₙ—O—), Cl, Br, I, OSO₂CF₃ or OSO₂(aryl); X′ is B(OR)₂, B(—O—(C(Rₐ)₂)ₙ—O—), Cl, Br, I, OSO₂CF₃ or OSO₂(aryl); each R is independently H, C₁-₃ alkyl or aryl; each Rₐ is independently C₁₋₃ alkyl or aryl; and each n is independently an integer of 2 or 3, wherein when X is B(OR)₂ or B(—O—(C(Rₐ)₂)ₙ—O—); X′ is Cl, Br, I, OSO₂CF₃ or OSO₂(aryl).

In related embodiments of the process for the preparation of the compound of formula (III), P₁ is H, (C═O)—C₁-C₈ alkyl, (C═O)-aryl or CH₂Aryl; P₂ is H, Et, or an amino protecting group selected from (C═O)—C₁-C₈ alkyl, (C═O)-aryl or (C═O)—O—C₁-C₈ alkylaryl; X is B(OR)₂ and B(—O—(C(Rₐ)₂)ₙ—O—); X′ is Cl, Br, I or OSO₂CF₃; R is independently H, C₁₋₃ alkyl or aryl; each Rₐ is independently C₁₋₃ alkyl or aryl; and n is an integer of 2 or 3.

In some embodiments of the process for the preparation of the compound of formula (III), P₁ is H, (C═O)—C₁-C₈ alkyl or CH₂Aryl; P₂ is H, Et, or an amino protecting group selected from (C═O)—C₁-C₈ alkyl, (C═O)-aryl and (C═O)—O—C₁-C₈ alkyl; X is B(OR)₂ or B(—O—(C(Rₐ)₂)ₙ—O—); X′ is Cl, Br, I or OSO₂CF₃; R is H, Rₐ is CH₃, and n=2.

In certain embodiments for the process for the preparation of the compound of formula (III), the process is conducted in the presence of a transition metal catalyst, said transition metal catalyst containing Pd (II), Cu (0) or Pd (0), or any combinations thereof.

In related embodiments for the process of preparation of the compound of formula (III) said transition metal catalyst is selected from the group consisting of Pd(OAc)₂, Pd(PPh₃)₄, PdCl₂(PPh₃)₂, Pd(dppf)Cl₂, Pd₂(Dba)₃, Cu(0) and Pd(PCy₃)₂, and any combinations thereof.

In some embodiments for the process of preparation of the compound of formula (III), said process is conducted in the presence of an inorganic or organic base, or any combinations thereof.

In some embodiments for the process of preparation of the compound of formula (III), said process is conducted in the presence of an inorganic base. In some further embodiments the inorganic base is selected from NaOH, KOH, Na₂CO₃, K₂CO₃, Cs₂CO₃, NaHCO₃, KHCO₃, and CsHCO₃. In a further embodiment, the inorganic base is KHCO₃.

In some embodiments, the compounds of formula (I), (II) and (III) have the structures (a), (aa′) and (b).

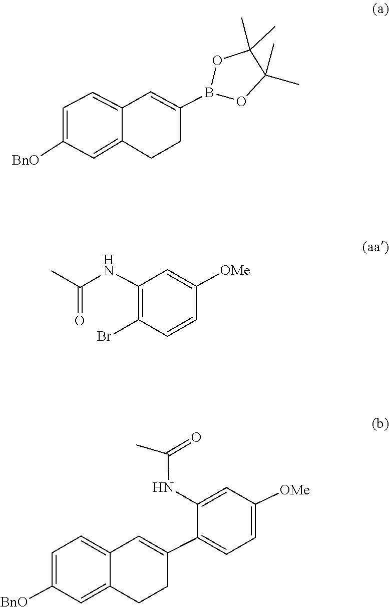

In some aspects, a process of preparing a compound of formula (IV) is described:

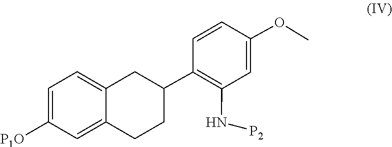

comprising the reduction of a compound of formula (III):

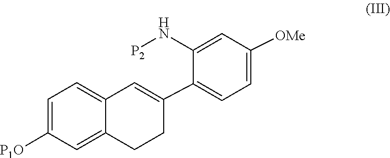

in the presence of a reducing agent, wherein:
P₁ is a H or a phenol protecting group and P₂ is H, Et or an amino-protecting group.

In some embodiments, the process of preparing a compound of formula (IV) is described wherein the reducing agent is H₂ in the presence of a transition metal catalyst.

In certain embodiments, the process of preparing a compound of formula (IV) is described wherein the transition metal catalyst contains Pd(II).

In some embodiments, the process of preparing a compound of formula (IV) is described wherein the transition metal catalyst is Pd(OH)₂.

In some embodiments, the process of preparing a compound of formula (IV) is described wherein P₁ is H, (C═O)—C₁-C₈ alkyl, (C═O)-aryl, (C═O)-heteroaryl, Si(C₁-C₅ alkyl)₃, Si(aryl)₂(C₁-C₅ alkyl) or CH₂Aryl and P₂ is H, Et, or an amino protecting group selected from (C═O)—C₁-C₈ alkyl, (C═O)-aryl, (C═O)-heteroaryl, (C═O)—O—C₁-C₈ alkyl, (C═O)—O-aryl, (C═O)—O-heteroaryl, (C═O)—O—C₁-C₈ alkylaryl and —(C═O—(CH₂)ₙ—C═O).

In certain embodiments, the process of preparing a compound of formula (IV) is described wherein P₁ is H, (C—O)—C₁-C₈ alkyl or CH₂Aryl; P₂ is Et, or an amino protecting group selected from (C═O)—C₁-C₈ alkyl, (C═O)-aryl and (C═O)—O—C₁-C₈ alkyl.

In certain embodiments, the process of preparing a compound of formula (IV) is described wherein P₁ is CH₂Ph and P₂ is (C═O)—CH₃.

In certain embodiments, the process of preparing a compound of formula (IV) is described wherein P₁ is H and P₂ is (C═O)—CH₃.

In some aspects, a process of preparing a compound of formula (V) is provided:

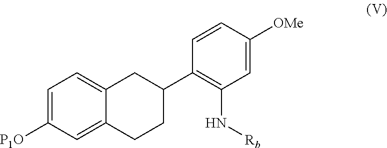

comprising the reaction of a compound of formula (IV):

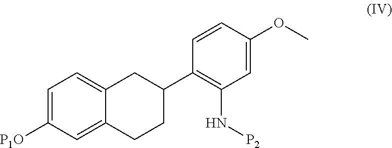

with an acid, base, nucleophile or reducing agent wherein:

P₁ is H or a phenol protecting group, P₂ is an amino-protecting group and R_{b} is H or Et.

In certain aspects, the process of preparing a compound of formula (V) is described wherein P₂ is (C═O)—CH₃ and R_{b} is Et and wherein the compound of formula (IV) is treated with a hydride-containing reducing agent. In some embodiments, the reducing agent is an inorganic reducing agent. In some embodiments the reducing agent is an inorganic hydride-containing reducing agent. In some further embodiments, the reducing agent contains boron or aluminum. In yet further embodiments, the reducing agent is a boron or aluminum hydride containing sodium, lithium or potassium. In some embodiments, the reducing agent is AlH₃, AlH₂Cl, AlHCl₂, NaBH₄, LiAlH₄, LiBH₄, LiEt₃BH, BH₃, BH₃. THF, CH₃CH₂C(O)OBH₃Na, Zn(OAc)₂/(EtO)₃SiH, Mg/TiCL₄, (HBpin)/tris(4,4-dimethyl-2-oxazolinyl)phenylborateMgMe, or combinations thereof.

In some aspects, a process of preparing a compound of formula (V) is provided:

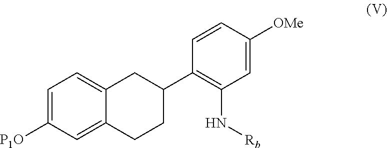

comprising the reaction of a compound of formula (IV):

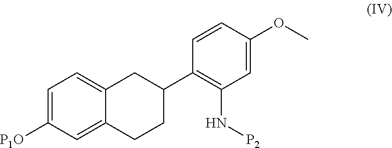

with an acid, base, nucleophile or reducing agent wherein:

P₁ is H or a phenol protecting group, P₂ is (C═O)—CH₃ and R_{b} is H or Et.

In certain aspects, the process of preparing a compound of formula (V) is described wherein P₂ is (C═O)—CH₃ and R_{b} is Et and wherein the compound of formula (IV) is treated with a hydride-containing reducing agent. In some embodiments, the reducing agent is an inorganic reducing agent. In some embodiments the reducing agent is an inorganic hydride-containing reducing agent. In some further embodiments, the reducing agent contains boron or aluminum. In yet further embodiments, the reducing agent is a boron or aluminum hydride containing sodium, lithium or potassium. In some embodiments, the reducing agent is AlH₃, AlH₂Cl, AlHCl₂, NaBH₄, LiAlH₄, LiBH₄, LiEt₃BH, BH₃, BH₃. THF, CH₃CH₂C(O)OBH₃Na, Zn(OAc)₂/(EtO)₃SiH, Mg/TiCL₄, (HBpin)/tris(4,4-dimethyl-2-oxazolinyl)phenylborateMgMe, or combinations thereof.

In certain aspects, the process of preparing a compound of formula (V) is described wherein P₂ is (C═O)—CH₃ and R_{b} is H and wherein the compound of formula (IV) is treated with acid, in some embodiments, the acid is hydrochloric acid or another protic acid.

In certain aspects, the process of preparing a compound of formula (V) is described wherein the compound according to formula (IV) is treated with an acid, base or nucleophile, and R_{b} is H.

In certain aspects, the process of preparing a compound of formula (V) is described wherein the compound according to claim (IV) is treated with an acid or base, in some embodiments said acid or base is in an aqueous solution.

In some embodiments, P₂ is (C═O)—C₁-C₈ alkyl, (C═O)-aryl, (C═O)-heteroaryl, (C═O)—O—C₁-C₈ alkyl, (C═O)—O-aryl, (C═O)—O-heteroaryl, (C═O)—O—C₁-C₈ alkylaryl or (C═O—(CH₂)ₙ—C═O). In some embodiments, P₂ is (C═O)—CH₃.

In some embodiments, P₁ is H, (C═O)—C₁-C₈ alkyl, (C═O)-aryl, (C═O)-heteroaryl, Si(C₁-C₅ alkyl)₃, Si(aryl)₂(C₁-C₅ alkyl) or CH₂Aryl. In some embodiments, P₁ is H or —CH₂—C₆H₅.

In yet other aspects, a process for increasing the enantiomeric excess of a

compound of formula (VI) is described:

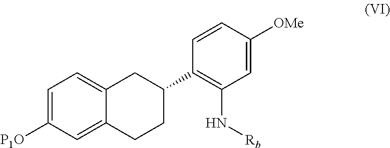

comprising the formation of a diastereomeric acid addition of the mixture (V):

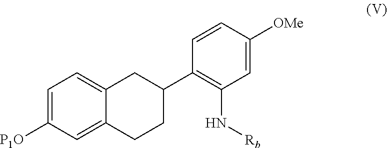

and selectively crystallizing the diastereomeric salt enhanced with the enantiomer of formula (VI) and subsequently freeing the base wherein P₁ is H or a phenol protecting group and R_{b} is H or Et. In some embodiments, the enantiomeric excess is >10%, >50%, >75%, >90%, >95%, >98% or >99%.

In another aspect, a process for increasing the enantiomer excess of a compound of formula (VI) is described:

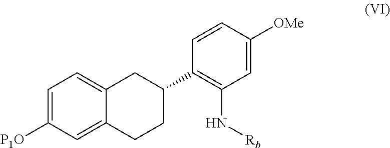

comprising contacting a compound of formula (V) with a chiral acid to form an enantiomerically enriched salt,

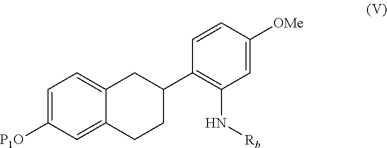

crystallizing the enantiomerically enriched salt, and freeing the compound of formula (VI), wherein P₁ is H or a phenol protecting group; and R_{b} is H or Et.

In some aspects, the process of enhancing the enantiomeric excess of a compound of formula (VI) utilizes a (+) or (−) chiral acid selected from Aspartic acid, O-Acetyl-Mandelic acid, cis-2-Benzamidocyclohexanecarboxylic acid, 1,1′-Binapthyl-2,2′-diyl hydrogen phosphate, Camphoric acid, 10-Camphorsulfonic acid, trans-1,2-Cyclohexanedicarboxylic acid, Dibenzoyl-Tartaric acid, Diacetyl-tartaric acid, Di-p-toluoyl-tartaric acid, N-(3,5-Dinitrobenzoyl)-α-phenylglycine, Diacetyl-tartaric anhydride, Diacetyl-tartaric acid, Glutamic acid, Malic acid, Mandelic acid, N-(α-methylbenzyl)phthalamic acid, 2-(6-Methoxy-2-napthyl)propionic acid, Pyroglutamic acid, Quinic acid and Tartaric acid, or combinations thereof. In some embodiments the acid is (+)-2,3-Dibenzoyl-D-tartaric acid.

In some embodiments, for the process of enhancing the enantiomeric excess of a compound of formula (VI); P₁ is H, (C═O)—C₁-C₈ alkyl, (C═O)-aryl, (C═O)-heteroaryl, Si(C₁-C₅ alkyl)₃, Si(aryl)₂(C₁-C₅ alkyl) or CH₂Aryl. In certain embodiments, P₁ is H, (C═O)—C₁-C8 alkyl or CH₂Aryl. In some embodiments, P₁ is —CH₂Ph or H. In some further embodiments, P₁ is H. In some embodiments, P₁ is H and P₂ is H.

In certain embodiments, a process of preparing a compound of formula (VII) is described:

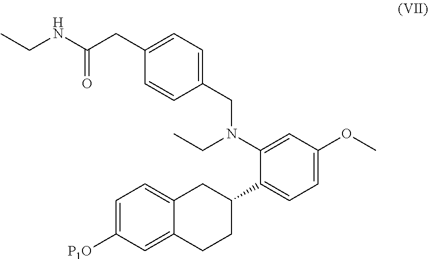

wherein P₁ is H or a phenol protecting group; comprising the reaction of a compound of formula (f):

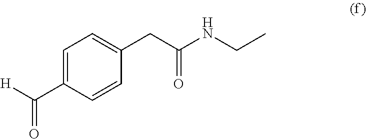

with a compound of formula (VI):

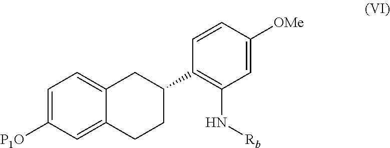

in the presence of a reducing agent wherein P₁ is H or a phenol protecting group and R_{b} is H or Et. In some embodiments, P₁ is H and R_{b} is H.

In some embodiments wherein R_{b} is H, said reducing agent is NaBHₙ(OAc)_{n′}; wherein n is an integer from 1-3, n′ is an integer from 1-3, and n+n′=4. In some embodiments, NaBHₙ(OAc)_{n′} is NaBH(OAc)₃.

In certain embodiments, R_{b} is Et and said reducing is an inorganic hydride-containing reducing agent. In some embodiments, said inorganic hydride-containing reducing agent contains boron or aluminum. In further embodiments, said reducing agent is a boron or aluminum hydride containing sodium, lithium or potassium. In some embodiments said reducing agent is AlH₃, AlH₂Cl, AlHCl₂, NaBH₄, LiAlH₄, LiBH₄, LiEt₃BH, BH₃, BH₃. THF, CH₃CH₂C(O)OBH₃Na, Zn(OAc)₂/(EtO)₃SiH, Mg/TiCL₄, (HBpin)/tris(4,4-dimethyl-2-oxazolinyl)phenylborateMgMe, or combinations thereof.

In some embodiments of the process of preparing a compound of formula (VII), P₁ is H.

In some embodiments of formula (VIII), P₁ is H, and the compound of formula (VIII) is Compound 1.

In certain embodiments, a process of preparing a compound of formula (VIII) is described:

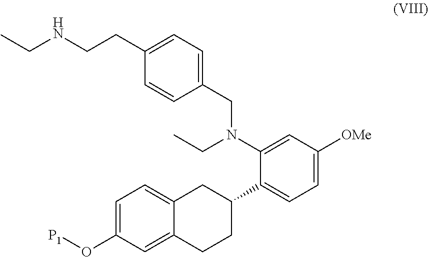

comprising the reduction of a compound of formula (VII):

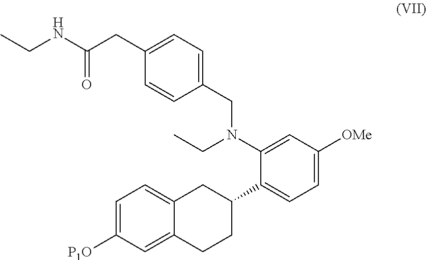

wherein P₁ is H or a phenol protecting group.

In certain embodiments of the process, the reduction is done in the presence of AlH₃, AlH₂Cl, AlHCl₂, NaBH₄, LiAlH₄, LiBH₄, LiEt₃BH, BH₃, BH₃. THF, CH₃CH₂C(O)OBH₃Na, Zn(OAc)₂/(EtO)₃SiH, Mg/TiCL₄, (HBpin)/tris(4,4-dimethyl-2-oxazolinyl)phenylborateMgMe or combinations thereof, and in some embodiments, the process is conducted in the presence of in situ generated BH₃ and in some embodiments, the reaction is conducted in the presence of NaBH₄/I₂.

In some embodiments, a process of preparing a compound of formula (VIII) is described:

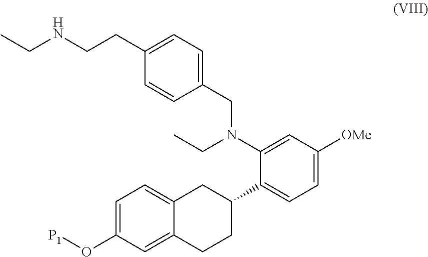

comprising the reduction of a compound of formula (VII);

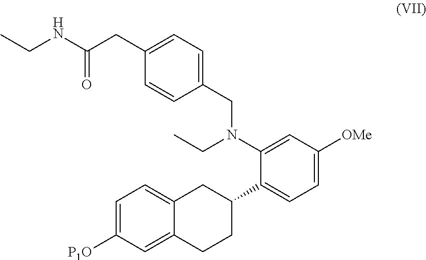

wherein P₁ is H, or a phenol protecting group; and wherein said compound of formula (VII) is prepared by a reductive amination of a compound of formula (VI);

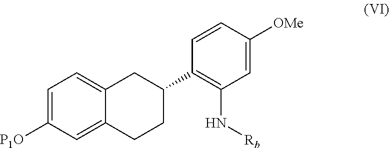

wherein R_{b} is Et or H and P₁ is hydrogen or a phenol protecting group; in the presence of a compound of formula (f);

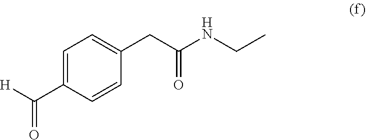

wherein said compound of formula (VI) is prepared by crystallization of a diastereomeric acid addition salt of a mixture having the formula (V) wherein R_{b} is Et or H and P₁ is hydrogen or a phenol protecting group;

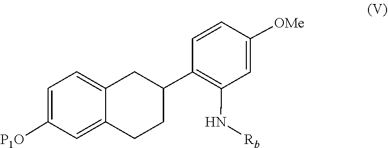

wherein said compound of formula (V) is prepared by treatment of a compound of formula (IV) with acid, base or a nucleophile when P₂ is (C═O)—C₁-C₈ alkyl, (C═O)-aryl, (C═O)-heteroaryl, (C═O)—O—C₁-C₈ alkyl, (C═O)—O-aryl, (C═O)—O-heteroaryl, (C═O)—O—C₁-C8 alkylaryl or —(C═O—(CH₂)ₙ—C═O), or optionally a reducing agent when P₂ is (C═O)—CH₃ and wherein P₁ is hydrogen or a phenol protecting group;

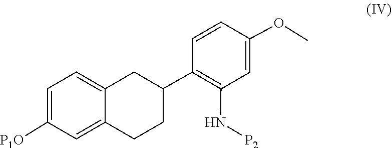

wherein said compound of formula (IV) is prepared by reduction of a compound of formula (III) wherein P₁ is H or a phenol protecting group and P₂ is H, Et, or an amino protecting group selected from (C═O)—C₁-C₈ alkyl, (C═O)-aryl, (C═O)-heteroaryl, (C═O)—O—C₁-C₈ alkyl, (C═O)—O-aryl, (C═O)—O-heteroaryl, (C═O)—O—C₁-C₈ alkylaryl and (C═O—(CH₂)ₙ—C═O);

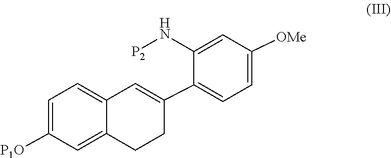

wherein said compound of formula (III) is prepared by the coupling of a compound of formula (I) wherein P₁ is H or a phenol protecting group, and X is a halogen, transition metal or boron-containing compound;

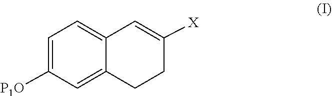

with a compound of formula (II) wherein X′ is a halogen, transition metal-containing function or boron-containing function and P₂ is H, Et or an amino-protecting group:

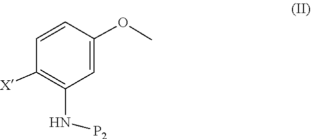

and wherein said X and X′ are suitable for cross-coupling of compound (I) with compound (II).

In one aspect, a process of preparing a compound of formula (VIII) is described,

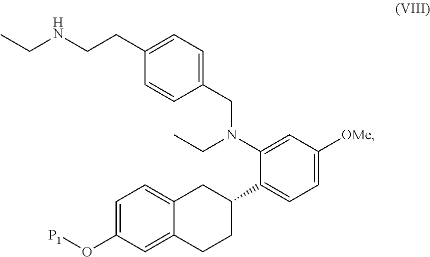

comprising the reduction of a compound of formula (VII)

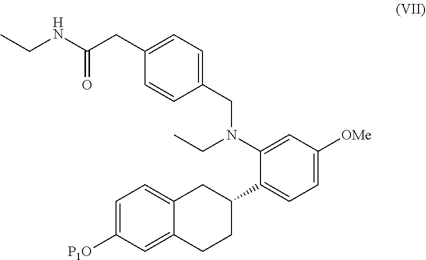

wherein said compound of formula (VII) is prepared by a reductive amination of a compound of formula (VI)

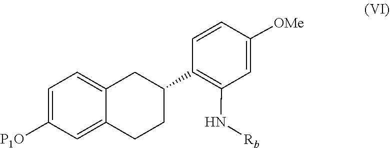

in the presence of a compound of formula (f)

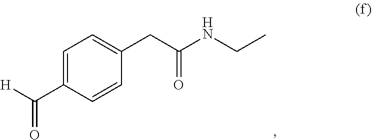

wherein said compound of formula (VI) is prepared by contacting a compound of formula (V)

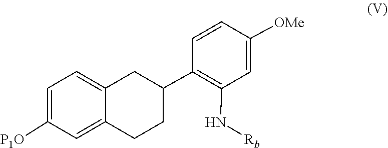

with a chiral acid to form an enantiomerically enriched salt, crystallizing the enantiomerically enriched salt, and freeing the compound of formula (VI),
wherein said compound of formula (V) is prepared by treatment of a compound of formula (IV) with a reducing agent;

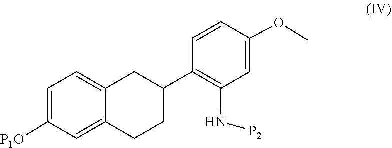

wherein said compound of formula (IV) is prepared by reduction of a compound of formula (III)

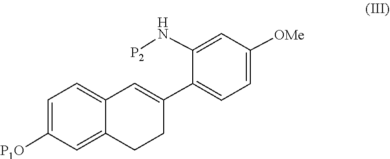

wherein said compound of formula (III) is prepared by the coupling of a compound of formula (I)

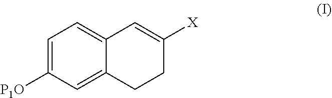

with a compound of formula (II)

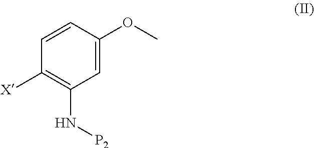

and wherein P₁ is H, or a phenol protecting group; R_{b} is H or Et; P₂ is (C═O)—CH₃; X is a halogen, transition metal or boron-containing compound; and X′ is a halogen, transition metal-containing function or boron-containing function; and wherein said X and X′ are suitable for cross-coupling of compound (I) with compound (II).

In some embodiments of the process of preparing a compound of formula (VII), P₁ is H. In some embodiments of formula (VIII), P₁ is H, and the compound of formula (VIII) is Compound 1.

### DETAILED DESCRIPTION OF THE INVENTION

Compound 1 has demonstrated promising mixed activity as a selective estrogen receptor alpha (ERα) modulator/degrader (SERM/SERD) against breast cancer, acting as a SERM at low doses and a SERD at high doses.

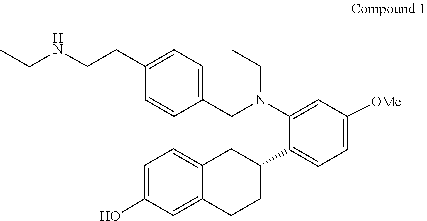 [SMILES: CCNCCc1ccc(CN(CC)c2cc(OC)ccc2[C@@H]2CCc3cc(O)ccc3C2)cc1]

Due to the interest in further development of Compound 1, larger quantities derived from innovative and efficient syntheses are needed for both preclinical and clinical studies with the eventual hope that the compound will be approved for commercial use, after which even much larger amounts will need to be produced. Accordingly, new and more efficient syntheses are needed. The present disclosure provides new and unexpectedly improved syntheses over the prior art disclosures (see e.g., U.S. Pat. No. 7,612,114).

The present disclosure provides both general procedures as well as specific examples demonstrating the efficacy of the described procedures.

As used herein, the terms below have the following definitions unless stated otherwise.

“Compound 1” or “RAD1901” has the following structure:

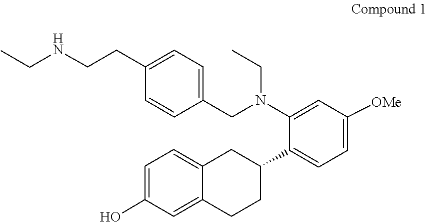 [SMILES: CCNCCc1ccc(CN(CC)c2cc(OC)ccc2[C@@H]2CCc3cc(O)ccc3C2)cc1]

including salts, solvates (e.g. hydrate), and prodrugs thereof. In some embodiments, the pharmaceutically acceptable salt of Compound 1 is “Compound 1 dihydrochloride” or “Compound 1 bis hydrochloride (·2HCl) salt” having the following structure:

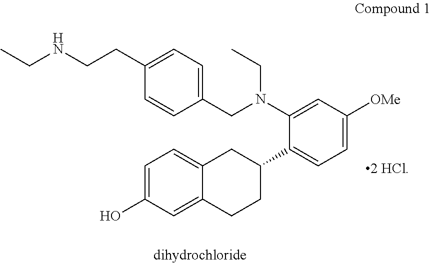 [SMILES: CCNCCc1ccc(CN(CC)c2cc(OC)ccc2[C@@H]2CCc3cc(O)ccc3C2)cc1]

A “halogen” atom is a fluorine, chlorine, bromine or iodine.

An “alkyl” group is a linear or branched chain, monovalent saturated hydrocarbon radical optionally substituted with up to five independently selected halogen atoms, hydroxyl groups (—OH), methyl, ethyl or propyl ether groups (—OMe, —OEt, —OPr or —OiPr), cyano groups (—CN) or —NO₂ groups. For example, a C₁₋₅ alkyl group includes -methyl, -ethyl, -isopropyl, -2-chloro-3-hydroxylbutyl, -2-fluoro-4-nitro-pentyl, etc.

An “aryl” group is a monovalent aromatic hydrocarbon radical of 6-20 carbon atoms (C₆-C₂₀). Aryl includes such structures as phenyl, biphenyl, naphthyl, etc. Aryls can be optionally substituted with up to five substituents independently selected from -halogen, -C₁₋₆ alkyl ethers, -hydroxyl, —CN, —C₁₋₆ alkyl and —NO₂.

A “heteroaryl” group is a cyclic aromatic group containing between 4 and 9 carbon atoms and containing between 1 and 3 heteroatoms, such as nitrogen, oxygen or sulfur. Said heteroaryl group may be monocyclic or bicyclic. By way of non-limiting example said heteroaryl includes without limitation oxazole, pyridine, quinoline, pyran, pyrrole and the like. Further, said heteroaryl can be substituted with up to five substituents selected from -halogen, —C₁₋₆ alkyl ethers, -hydroxyl, —CN, —C₁₋₆ alkyl and —NO₂.

Where terms are joined, as in, for example, —C₁-C₈ alkylaryl, the definitions for the separate functions (e.g., “C₁₋₈ alkyl” and “aryl”) are each as defined separately including, for example, substitutions and branching. Thus, a C₁₋₈ alkylaryl could include an alkyl radical of 1-(3-chloro-nitrobutyl)-3-methylbenzene where the radical point of attachment is on the terminus of the butyl fragment as shown below:

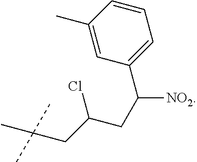

In the processes provided herein, reference is made to protecting groups, such as “a phenol protecting group” or an “amino protecting group”. When described accordingly, one of ordinary skill in the art will appreciate that the particular protecting group can be selected from protecting groups known to those of skill in the art and also protecting groups varying from those known to those of skill but understood to be logical extensions of those groups and understood or predicted to operate by the same mechanism and having similar properties to those most closely related to those known in the art. While not wishing to be bound by example, protecting groups useful for the processes outlined herein can be found in various textbooks and hereby incorporated by reference. For example, see Protective Groups in Organic Synthesis (Green (Wuts), Wiley Publishing), Protecting Groups in Organic Synthesis: (Postgraduate Chemistry Series) (Hanson, Wiley Publishing), Protecting Groups (Kocienski, Thieme Publishing).

An example of the processes described here is shown in Scheme 1 and described briefly below. The vinyl bromide exemplified by compound (a′) can be converted to its boron derivative (a) (2-(6-(benzyloxy)-3,4-dihydronaphthalen-2-yl)-4,4,5,5-tetramethyl-1,3,2-dioxaborolane) and then coupled with (aa′) (N-(2-bromo-5-methoxyphenyl)acetamide) in the presence of base to render the coupled compound (b) (N-(2-(6-(benzyloxy)-3,4-dihydronaphthalen-2-yl)-5-methoxyphenyl) acetamide in an overall yield of >70%. Compound (b) was then reduced with Pd(OH)₂/H₂ which both reduced the double bond and debenzylated the phenol to render (c) which was subsequently de-acetylated to produce compound (d) with the yield over both steps (b to d) of >90%. Compound (d) is typically a 50:50 racemic mixture and the desired compound has the (R)-stereochemistry at the 6-position. In this regard, it has been discovered that forming the acid addition salt with the appropriate chiral acids (as described herein) followed by crystallizing the product can efficiently increase the enantio-purity of compound (e) and related derivatives. In the present example, a racemic mixture was treated with (+)-2,3-dibenzoyl-D-tartaric acid [(+)-DBTA, 0.5 eq.] and the desired salt crystallized out in >90% ee and >90% of the theoretical yield of the desired enantiomer. The next step is the reductive amination of compound (e) with benzaldehyde (f) wherein the Schiff base is formed first between the aldehyde (f) with the aniline (e), followed by reduction with NaBH(OAc)₃ and the yield of crude product >90%. The reaction reduces the Schiff base formed between the amine (e) and benzaldehyde (f) and surprisingly also ethylates the aniline to give the desired tertiary aniline. Not wishing to be bound by theory, it is believed that acetyl transfer and reduction from the NaBH(OAc)₃ reagent occurs. The product (g) was reduced with NaBH₄/I₂ which is believed to generate BH₃ in situ followed by a reductive work up with Na₂S₂O₃ and the product purified and treated with HCl (MeOH, EtOH and/or EtOAc) and the yield of the bis HCl salt of Compound 1 was >50%.

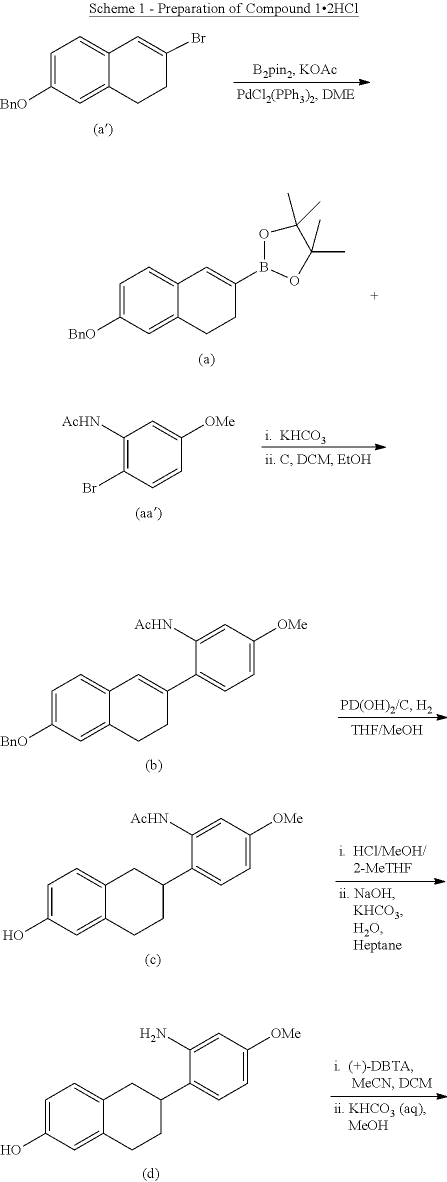 [SMILES: BrC1=Cc2ccc(OCc3ccccc3)cc2CC1 ; CC1(C)OB(C2=Cc3ccc(OCc4ccccc4)cc3CC2)OC1(C)C ; COc1ccc(Br)c(NC(C)=O)c1 ; COc1ccc(C2=Cc3ccc(OCc4ccccc4)cc3CC2)c(NC(C)=O)c1 ; COc1ccc(C2CCc3cc(O)ccc3C2)c(NC(C)=O)c1 ; COc1ccc(C2CCc3cc(O)ccc3C2)c(N)c1]

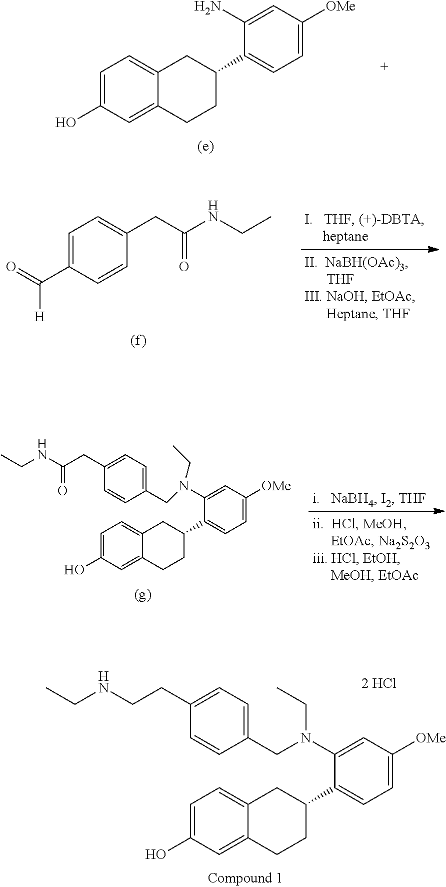

Step 1—Preparation of N-(2-(6-(benzyloxy)-3,4-dihydronaphthalen-2-yl)-5-methoxyphenyl)acetamide (b).

A solution of (a′, 1 eq.) and bis(pinacolato)diboron (1.3 eq.) were dissolved in 7 volumes of 1,2-dimethoxyethane (DME), treated with KOAc (3.1 eq.) and PdCl₂(PPh₃)₂ (2 mol %), and heated at 85° C. The reaction was then monitored for completion by HPLC. The solution was cooled to 20° C. and treated with 25 wt % KHCO₃ (aqueous, 3 volumes) and 2-bromo-5-methoxyacetanilide (aa′, 1 eq), then heated to 85° C. and monitored for completion by HPLC. The reaction was then cooled to 55° C. and the mixture filtered and the solids washed with DME. The water layer was separated off and the remaining organic layer cooled to 20° C. and diluted with 6.9 volumes of water. The mixture was then agitated for ≥1 hour and the formed solids filtered and washed with 3.1 volumes of water and the cake dried at ≤55° C. The combined solids were treated with 10 volumes of dichloromethane (DCM) and carbon (0.25 wt equivalents) and the resulting mixture was stirred and heated to reflux (approx. 40° C.) for ≥6 hours. The mixture was then cooled to 20° C., the solid filtered, and washed with 3 volumes of DCM. The resulting filtrate solution was concentrated to 3 volumes under vacuum at ≤45° C. and treated with 6 volumes of ethanol (EtOH) and concentrated under vacuum at ≤45°° C. to 4.9 volumes. An additional 6 volumes of EtOH was added and the volume once again concentrated under vacuum at ≤45° C. to 4.9 volumes and 1 volume of EtOH added and cooled to 20° C. and the product collected by filtration and rinsed with 1 volume of EtOH and dried under N₂ at ≤50°° C. to provide (b) in >70% yield.

Step 2—Preparation of (+/−)N-(2-(6-hydroxy-1,2,3,4-tetrahydronaphthalen-2-yl)-5-methoxypheny)acetamide (c)

A solution of (b) (1 eq.), Pd(OH)₂/C (0.1 weight equivalents), THF (7 volumes) and MeOH (7 volumes) was purged with N₂ and then H₂ at 20° C. The reaction mixture was agitated under 100 psi H₂ for ≥12 hours at 20° C. and the reaction monitored for completion. After purging the reaction with N₂ at 20° C., the reaction mixture was heated at 40° C. for ≥1 hour, filtered and rinsed with 1.5 volumes of THF and 1.5 volumes of MeOH. The solution was concentrated to 2.4 volumes under vacuum at ≤45° C. and treated with 12 volumes of EtOAc, concentrated to 2.4 volumes under vacuum at ≤45° C., treated again with 12 volumes of EtOAc and concentrated to 2.4 volumes under vacuum at ≤45° C. and treated with 3.3 volumes of EtOAc and the temperature adjusted to 20° C. and agitated at 20° C.≥1 hour, the product collected by filtration and washed with 1.4 volumes of EtOAc. The solid can be recrystallized in MeOH/EtOAc if desired and the product (c) dried at temperatures ≤50° C.

Step 3—Preparation of (+/−)6-(2-amino-4-methoxyphenyl)-5,6,7,8-tetrahydronapthalen-2-ol (d)

A solution of ((c), 1 eq.) in 9 volumes of MeOH and concentrated HCl (1.5 wt equivalents) was heated and agitated at reflux ≥16 hours (approx. 65° C.) and monitored for completion. The reaction was cooled to ≤35° C. and concentrated under vacuum at ≤45° C. to 3.8 volumes, charged with 3 volumes of 2-MeTHF and concentrated under vacuum at ≤45° C. to 3.8 volumes, charged with 3 volumes of 2-MeTHF and concentrated under vacuum at ≤45° C. to 3.8 volumes, charged with 12 volumes of 2-MeTHF then 10 volumes of 1 M NaOH followed by 1.5 weight equivalents of 25% KHCO₃ while maintaining the internal temperatures at ≤35° C. The internal temperature was adjusted to 20° C. and stirred for ≥15 minutes. The pH was adjusted/maintained to between 8-10 using 1 M HCl or 1 M NaOH. The agitation was stopped and the aqueous layer separated out after settling and 1 volume of H₂O added and the solution stirred ≥15 minutes and the aqueous layer removed after settling and one additional volume of H₂O added and the aqueous layer separated out after settling. The organic layer was concentrated under vacuum to 3 volumes at ≤45° and the solution treated with 3 volumes of heptane and agitated for 12 hours at 20° C. The solids were collected by filtration and the filter cake rinsed with 2 volumes of heptane. The solvents were evaporated at ≤50° C. to provide the title compound (d) in a yield of >90%.

Step 4—Preparation of compound (R)-6-(2-amino-4-methoxyphenyl)-5,6,7,8-tetrahydronapthalen-2-ol (e)

One equivalent of (d) in 14.2 volumes of MeCN and 4.8 volumes of DCM was heated to 40° C. To this was added (+)-2,3-dibenzoyl-D-tartaric acid [(+)-DBTA, 0.5 eq.] and heated to reflux (approximately 65° C.). The reactor was cooled to 50° C. for approximately 1 hour, cooled to 40° C. for approximately 1 hour and cooled to 25° C. for approximately 1 hour. The slurry was filtered and the filter cake washed with 2 volumes of DCM. The wet filter cake was refluxed (approximately 44° C.) in 8 volumes of DCM for ≥1 h. The solution was cooled to 25° C. at a rate of 15° C./h and stirred at 25° C. for ≥1 hour. The slurry was filtered and washed with 2 volumes of DCM and the cake was again slurried with 8 volumes of DCM for 1 hour at ambient temperature and then filtered and washed with 2 volumes of DCM and dried. (The chiral purity was assayed at this point, providing an enantiomeric excess of >90%).

A solution containing the (+)-DBTA salt, 15 volumes of water and 3 volumes of methanol was treated with 4.6 volumes of a 25% KHCO₃ aqueous solution and agitated at 25° C. for ≥1 h. The solids were collected by filtration and rinsed with 4 volumes of water. The aqueous layer was adjusted to a pH of ≥8 using 25% KHCO₃ as needed, and the resulting solids were collected by filtration. The filter cake was washed with 4 volumes of water. The combined solids were added to 4 volumes of water, the resulting slurry was stirred for ≥1 h, and the solids were then collected by filtration. The filter cake was washed with 4 volumes of water and 4 volumes of heptane and was dried at ≤50° C. to provide the title compound (R)-6-(2-amino-4-methoxyphenyl)-5,6,7,8-tetrahydronapthalen-2-ol (e). The yield was determined to be >90% and the ee was determined to be >90%.

Step 5-Preparation of (R)-N-ethyl-3-(4-((ethyl(2-(6-hydroxy-1,2,3,4-tetrahydronapthalen-2-yl)-5-methoxyphenyl)amino)methyl)propanamide (g)

A mixture containing 1 equivalent of compound (e) together with 1 wt equivalent of activated molecular sieves and anhydrous THF was agitated at ambient temperature for ≥2 hours. The mixture was filtered through THF-compacted celite and rinsed with 10 volumes of THF. The solution was charged with N-ethyl-2-(4-formylphenyl)acetamide (f) (1.2 eq.) and 7.5 volumes of heptane and DBTA (0.1%) and heated to reflux (approximately 65° C.). The mixture was atmospherically distilled to 10 volumes at reflux. The reaction was monitored for completion via TLC. 2.9 volumes of heptane and 7.1 volumes of THF were added and the reaction was atmospherically distilled to 10 volumes at reflux and monitored for completion by TLC. The solution was cooled to 20° C. and agitated for ≥5 hours to ensure that crystallization had occurred. The solid product was collected by filtration, rinsed with 2 volumes of heptane and dissolved in 40 volumes of anhydrous THF and treated with 4.5 equivalents of NaBH(OAc)₃. The mixture was heated to 50° C. for ≥16 hours and monitored by TLC. An additional 4.5 equivalents of NaBH(OAc)₃ was added (additional N-ethyl-2-(4-formylphenyl)acetamide (f) could be added at this point if the reaction was not complete). The reaction was cooled to 20° C. and quenched with 15 volumes of 3 M NaOH. The solution/mixture was agitated for ≥30 minutes and the pH adjusted to 8-9 with 9% aqueous NaHCO₃ (approximately 14 volumes) if necessary. The aqueous layer was separated out and the organic layer concentrated to 5 volumes under vacuum at ≤45° C. The resulting solution was diluted with 10 volumes of EtOAc and concentrated to 5 volumes under vacuum at ≤45° C. The solution was treated with 10 volumes of EtOAc and 5 volumes of 5.6% NaCl solution, stirred and then allowed to settle and the aqueous layer removed. The mixture was dried with Na₂SO₄ (4 wt) and filtered and concentrated to 5 volumes under vacuum at ≤45° C., treated with 10 volumes of heptane and concentrated to 5 volumes under vacuum at ≤45° C., treated with 10 volumes of heptane and concentrated to 5 volumes under vacuum at ≤45° C. and treated with 10 volumes of heptane and concentrated to 5 volumes under vacuum at ≤45° C. The solution was then treated with 10 volumes of THF and dried under vacuum at ≤45° C. and treated again with 10 volumes of THF and dried to 5 volumes under vacuum at ≤45° C. and treated with 5 volumes of THF and residual heptane evaluated by GC (<4%) and the THE solution carried forward to the next reaction. The yield of the final product was determined to be >90%.

Step 6—Preparation of (R)-6-(2-(ethylamino)ethyl)benzyl)amino)-4-methoxyphenyl)-5,6,7,8-tetrahydronaphthalen-2-ol (Compound 1)

A reactor was charged with 7 volumes of THF and 2.5 equivalents of NaBH₄ and cooled to from −10° C. to 0° C. The solution was charged with the THE solution carried over from step 6 (1 equivalent of intermediate (g)) while maintaining the reactor temperature at ≤5° C. The solution was stirred with an internal temperature adjusted to −25° C. 1 Equivalent of I₂ in 1 volume of THF was added to the solution while maintaining the temperature at ≤−10° C. The mixture was agitated for ≥30 minutes at ≤−10° C. then heated to reflux and stirred at reflux (approximately 66° C.) for at 4 hours and monitored by HPLC for completion. The reaction mixture was cooled to ≤5° C. and quenched with 0.5 volumes of concentrated HCl while maintaining the reaction mixture temperature of ≤−10° C., and then treated with 15 volumes of water. The pH was checked and adjusted to less than 1.5 as needed. The solution was then heated to reflux and atmospherically distilled until the internal temperature reaches 80° C. The reaction mixture was cooled to 15-25° C., stirred for 6 hours and the solid isolated by filtration. The solid was charged back into the reactor along with 10 volumes of EtOAc and 5 volumes of 1 M NaOH and the mixture agitated for up to 30 minutes at 10-20° C. The pH was checked and adjusted to 8-9 as needed. The organic and aqueous layers were allowed to separate and the aqueous layer removed and washed with 10 volumes of EtOAc. The aqueous layer was removed and the combined organic layers were washed with 2×5 volumes of 5% sodium thiosulfate solution. The organic layer was washed with 4×10 volumes of 1% NaCl solution. The aqueous layer was removed and the organic layer concentrated to 3 volumes at an external temperature of up to 45° C. Three times, the residue was dissolved in 10 volumes of EtOH and concentrated to 3 volumes at ≤45° C. The solution was dried with Na₂SO₄ and filtered and the filtrate charged to a reactor where it was treated with 1 volume of EtOAc and stirred and charged with 3.3 M HCl in EtOH (1.4 volumes) and the mixture agitated at 15-25° C. for ≥2 hours and then concentrated to 4.6 volumes at ≤45° C. The solution was treated with 12.4 volumes of EtOAc and agitated at 15-25° C. for ≥2 hours to ensure that crystallization had occurred. The solids were collected by filtration and rinsed with 3.1 volumes of EtOAc. The filter cake was dried at ≤50° C. The material can be assayed for purity and recrystallized from MeOH/EtOAc if desired. The yield of the final product was >50% and the purity >90%. If desired, the product can be recrystallized from EtOH/EtOAc to generate a polymorphic form having good stability.

1. A compound selected from (a), (b), (e), (f), and (g)

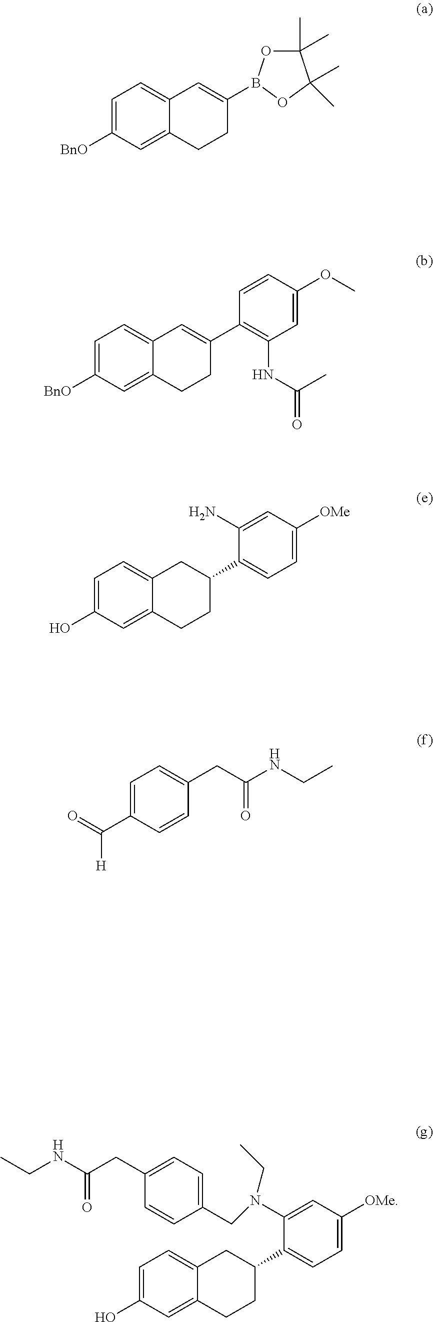

2. A compound of claim 1 having formula (e) or (g) wherein said compound is present in an enantiomeric excess of >50%.

3. A compound of claim 1 having formula (e) or (g) wherein said compound is present in an enantiomeric excess of >60%.

4. A compound of claim 1 having formula (e) or (g) wherein said compound is present in an enantiomeric excess of >70%.

5. A compound of claim 1 having formula (e) or (g) wherein said compound is present in an enantiomeric excess of >80%.

6. A compound of claim 1 having formula (e) or (g) wherein said compound is present in an enantiomeric excess of >90%.

7. A compound of claim 1 having formula (e) or (g) wherein said compound is present in an enantiomeric excess of >95%.

8. A compound of claim 1 having formula (e) or (g) wherein said compound is present in an enantiomeric excess of >98%.

9. A compound of claim 1 having formula (e) or (g) wherein said compound is present in an enantiomeric excess of >99%.

10-65. (canceled)

66. The compound according to claim 1, wherein the compound is Compound (a):

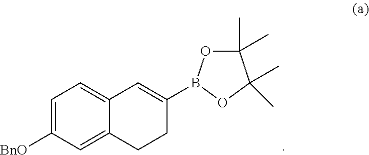 [SMILES: CC1(C)OB(C2=Cc3ccc(OCc4ccccc4)cc3CC2)OC1(C)C]

67. The compound according to claim 1, wherein the compound is Compound (b):

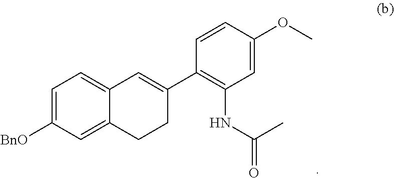 [SMILES: COc1ccc(C2=Cc3ccc(OCc4ccccc4)cc3CC2)c(NC(C)=O)c1]

68. The compound according to claim 1, wherein the compound is Compound (e):

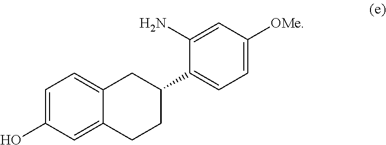 [SMILES: COc1ccc([C@@H]2CCc3cc(O)ccc3C2)c(N)c1]

69. The compound according to claim 1, wherein the compound is Compound (f):

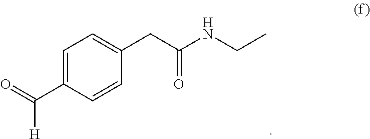

70. The compound according to claim 1, wherein the compound is Compound (g): 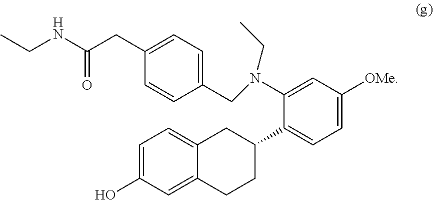 [SMILES: CCNC(=O)Cc1ccc(CN(CC)c2cc(OC)ccc2[C@@H]2CCc3cc(O)ccc3C2)cc1]
